(19) United States
(12) Patent Application Publication
(10) Pub. No.: US 2022/0105281 A1
(43) Pub. Date: Apr. 7, 2022

(54) WIRELESS GUM OR TISSUE NUMBING DEVICE

(71) Applicant: Natasha, LLC, Burbank, CA (US)

(72) Inventors: Ara Gulesserian, Glendale, CA (US); Alen Simon, Burbank, CA (US)

(73) Assignee: Natasha, LLC

(21) Appl. No.: 17/329,144

(22) Filed: May 25, 2021

Related U.S. Application Data
(60) Provisional application No. 63/088,733, filed on Oct. 7, 2020.

(51) Int. Cl. A61M 5/42; A61H 21/00; A61H 23/02

(52) U.S. Cl. CPC A61M 5/422; A61H 21/00; A61H 2201/5025; A61H 2201/5097; A61H 2201/0153; A61H 23/02

(57) ABSTRACT

A wireless gum numbing device includes an on or off switch, a soft rubber cover, and a base assembly with the soft rubber cover covering an underside of the base assembly. The wireless gum numbing device includes two lateral support assemblies, a finger holding clip and a fingertip retention piece. The two lateral side assemblies rise horizontally from a left side and a right side of the base assembly and hold a user's finger from moving laterally during use of the wireless gum numbing device. A finger of the user touches the finger holding clip. The fingertip retention piece connects the base assembly to the finger holding clip to prevent the fingertip from moving too far in a forward direction.

### RELATED APPLICATIONS

This application claims priority to and is related to U.S. provisional patent application Ser. No. 63/088,733, filed Oct. 7, 2020, entitled “Wireless Gum or Tissue Numbing Device,” the entirety of which is hereby incorporated by reference.

### BACKGROUND

The application of Novocain or other numbing agents to dental patients may be a painful procedure. Many dentists utilize a local numbing agent to numb the area where the Novocain shot may be administered. However, this application does not work in many cases because the local numbing agent does not work immediately, the local numbing agent may be applied to the wrong area because a nurse or hygienist is not familiar with where the dental work will be performed and/or the taste of the numbing agent may be transferred to the patient's throat due to saliva.

Accordingly, the Novocain shot may be painful and may take longer due to the discomfort felt by the patient. Tools have been developed to address this problem where a large device applies a vibration to the area where the Novocain shot will be delivered. However, the dentist is not able to pinpoint the large vibrating device with the greatest precision to the correct area because the vibration device is too large and is not flexible.

Therefore, a need exists for a numbing device that is flexible, small and able to numb an area where a needle will be inserted into a patient's tissue area (e.g., a gum area).

### BRIEF DESCRIPTION OF DRAWINGS

FIG. 1 illustrates wireless gum or tissue numbing devices according to some embodiments;

FIG. 2A illustrates a wireless gum or tissue numbing device inserted on a professional's finger according to some embodiments;

FIG. 2B illustrates an exploded view of the wireless gum or tissue numbing device according to some embodiments;

FIG. 3 illustrates an additional embodiment of the wireless gum or tissue numbing device according to some embodiments;

FIG. 4 illustrates an exploded view of an additional embodiment of the wireless gum or tissue numbing device according to some embodiments; and

FIG. 5 illustrates a control assembly for one or more wireless gum and tissue numbing devices according to some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein. Accordingly, a device is needed that may be flexible and/or may reach into small spaces. In some embodiments, a wireless gum or tissue numbing device may be placed and/or positioned onto a finger or fingertip of a medical professional so that the medical professional can easily apply it to any areas of the patient's gum and/or tissue. In some embodiments, a medical professional can utilize two wireless gum or tissue numbing devices in order to isolate a specific area of a patient's gum or tissue in between the gum or tissue numbing devices. This is especially useful for dentists. Although this description focuses on Novocain, the techniques and devices described herein may be utilized with the application of botox through needles or other substances through needles.

Although the above and below paragraphs describe a dental procedure, the device described below may be utilized with other patient tissue areas where patients will be receiving shots and/or having needles inserted. For example, the apparatus and/or methods describe above is also applicable to other professionals (e.g., medical or cosmetic) that inject medications or liquids (e.g., botox or lidocaine) into a patient's or individuals tissue. The numbing device described below has equal application to these other uses also.

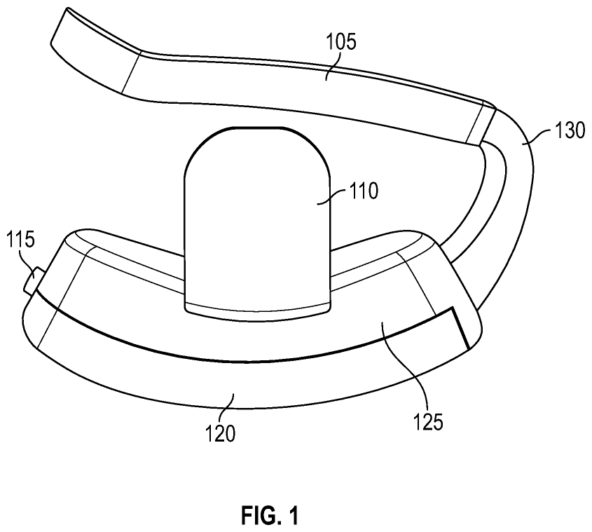

FIG. 1 illustrates wireless gum or tissue numbing devices according to some embodiments. In some embodiments, a wireless gum or tissue numbing device 100 may include a finger holding clip 105, a fingertip retention piece 130, a base plate piece 125, an over molded soft rubber cover assembly 120, one or more lateral finger support flaps or plates 110, and/or an on/off switch 115. The small footprint of the wireless gum or tissue numbing device allows a medical professional to utilize these devices in smaller areas than prior art devices have been utilized in. Most other devices are large devices that are utilized on larger or big areas of a patient, and thus cannot pinpoint an area in the mouth where a needle will be inserted into a patient's gum or a patient's tissue. The wireless gum or tissue numbing device may be placed on medical professional's fingers and thus take up less space. Because the device is placed on the medical professional's fingers, the device may easily be inserted inside a patient's mouth. In addition, because the wireless gum or tissue numbing device is positioned on a medical professional's fingers, the medical professional can localize and/or pinpoint where the numbing is applied and this provides the medical professional with a large amount of flexibility in administering the numbing injection.

In some embodiments, the finger holding clip 105 may be placed or positioned on a top of a medical professional's fingers. In some embodiments, a bottom surface of the finger holding clip 105 may touch or contact a top surface of the medical professional's fingers. In some embodiments, the finger holding clip 105 may range from 1.0 inches and 1.25 inches in length, or alternatively may range from 0.75 inches and 1.5 inches in length. In some embodiments, the finger holding clip 105 may range from 0.5 inches and 0.75 inches in width, or alternatively range from 0.3 and 0.9 inches in width. In some embodiments, the finger holding clip 105 may have a height ranging from 0.05 and 0.15 inches in height, or alternatively range from 0.025 and 0.0175 inches in height. In some embodiments, the finger holding clip 105 should have a small height to minimize an overall height footprint of the wireless gum or tissue numbing device 100. This small footprint allows the wireless gum or tissue numbing device to be utilized and/or applied in smaller spaces like a patient's mouth, although the claimed subject matter is not limited to this application. In some embodiments, a first end of the finger holding clip 105 is curved up or raised up with respect to a remainder of the body of the finger holding clip 105. In some embodiments, the second end of the finger holding clip 105 may be coupled to the fingertip retention piece 130. In some embodiments, the finger holding clip 105 may be made of a plastic or composite material, although other materials may be utilized.

In some embodiments, the fingertip retention piece 130 may be coupled to the finger holding clip 105 and may also be coupled to the base plate piece or assembly 125. In some embodiments, the fingertip retention piece 130 may be attached, coupled, connected to and/or inserted into a second end of the finger holding clip 105. In some embodiments, the fingertip retention piece 130 may be attached, coupled, connected and/or inserted into a second end of a base plate piece or assembly 125. In some embodiments, fingertip retention piece 130 may be positioned vertically between the finger holding clip 105 and the base place piece or assembly 125. In some embodiments, the fingertip retention piece 130 may be curved in shape to allow for the fingertip to be inserted (it follows the shape of the fingertip and has a longer opening on the portion of the fingertip retention piece 130 that is near the finger holding clip 105). In some embodiments, the fingertip retention clip 105 may be made of a plastic material and/or a composite material. In some embodiments, the fingertip retention clip may have a height ranging from 0.5 inches to 0.8 inches, or alternatively from 0.3 inches to 0.9 inches. In some embodiments, the fingertip retention clip 130 may have a width of 0.1 inches to 0.2 inches, or alternatively 0.05 inches to 0.25 inches.

In some embodiments, a second end of base plate piece or assembly 125 may be connected, attached, coupled and/or receive the fingertip retention piece 130. In some embodiments, an on/off switch and/or activation switch 115 may be connected, attached or coupled to, and/or embedded in a second end of the base place piece or assembly 125. In some embodiments, one or more lateral finger support flaps 110 may be connected, attached, coupled and/or inserted into the base plate piece or assembly 125. In the embodiment illustrated in FIG. 1, there are two lateral finger support flaps or support assemblies 110 that the medical professional's fingers may be inserted in between. In some embodiments, the one or more lateral finger support flaps or assemblies 110 may be connected, attached, or coupled to and/or inserted into opposite sides of the base plate piece or assembly 125. In some embodiments, the one or more lateral finger support flaps or assemblies 110 may have a curved shape where one end of the one or more lateral finger support flaps or assemblies 110 is inserted, coupled or attached to the base plate 125 and then the remainder of the one or more lateral finger support flaps or assemblies 110 is curved between 60 to 120 degrees so that the remainder and/or other end of the one or more lateral support flaps or assemblies 110 rise vertically from the sides of the base plate piece or assembly 125. In some embodiments, the one or more lateral finger support flaps or assemblies 110 are made of a plastic and/or composite material. In some embodiments, the medical professional may place their index finger and/or thumb in between the one or more lateral finger support flaps or assemblies 110. In some embodiments, the lateral finger support flaps or assemblies 110 may have a height range of 0.4 inches to 0.75 inches, or alternatively have a height range of 0.3 inches to 0.9 inches. In some embodiments, the lateral finger support flaps or assemblies 110 may have a width range of 0.3 inches to 0.5 inches, or alternatively may have a width range of 0.2 inches to 0.7 inches.

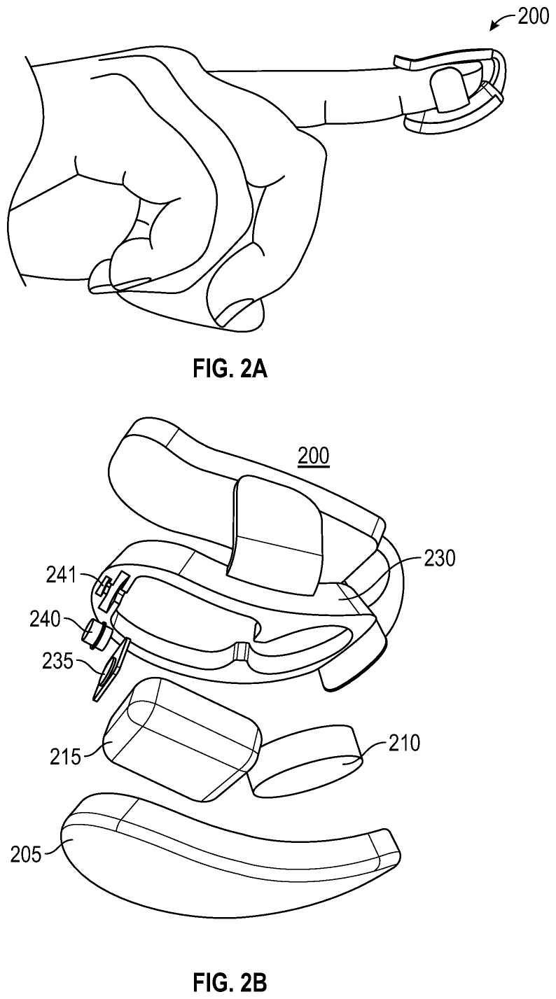

In some embodiments, the base plate piece or assembly 125 may have a shape that is curved upwards at both ends of the base plate piece or assembly 125. In some embodiments, by having a curved upward shape, the medical professional may always be able to apply or press as much of a bottom surface of the base plate piece or assembly 125 on the gum or tissue of the patient or user and thus have a device that provides the best performance (in terms of a surface area to be numbed). In some embodiments, the upwardly curved top surface of the base plate piece or assembly 125 mimics or approximates the shape of a bottom side of a medical professional's finger and/or fingertip. In some embodiments, the base plate piece or assembly 125 has a bottom side or underside where components may be located that are utilized to power and/or operate the wireless gum or tissue numbing device 100. As will be discussed with respect to FIGS. 2A and 2B, the bottom side of the base plate piece or assembly 125 includes one or more cavities for different components of the wireless gum numbing or tissue device. In some embodiments, the base plate piece or assembly 125 may have a ledge or lip or wall on the second end on its bottom side. In some embodiments, the base plate piece or assembly 125 may be made of a composite material and/or a plastic material. In some embodiments, an over molded soft rubber covering may cover a bottom surface of the base plate piece or assembly 125. In some embodiments, the over molded soft rubber covering 120 may hold the components in the openings or cavities of the base plate piece or assembly 125 and/or also protect these components. The over molded soft rubber covering 120 may be pressed against or in contact with an inside surface of the lip or ledge of the bottom side of the base plate piece or assembly 125. In some embodiments, the over molded covering 120 is made of rubber because it comes in contact with a patient's mouth, gums and/or tissue and thus cannot be rough or abrasive. In some embodiments, a base plate piece or assembly 125 may range in length from 1.05 inches to 1.45 inches, or alternatively from 0.80 to 1.6 inches. In some embodiments, a base plate piece or assembly 125 may range in height from 0.1 to 0.2 inches or from 0.05 inches to 0.3 inches. In some embodiments, a base plate piece of assembly 125 may have a width ranging from 0.58 to 0.85 inches, or alternatively from 0.50 to 0.95 inches. In some embodiments, the over molded soft rubber covering 120 may have a length slightly less that the base plate piece or assembly 125, may have a height that is less than the based plate piece or assembly 125, and/or a similar width to the base plate piece or assembly 125.

FIG. 2A illustrates a wireless gum or tissue numbing device inserted on a professional's finger according to some embodiments. FIG. 2B illustrates an exploded view of the wireless gum or tissue numbing device according to some embodiments. FIG. 2A illustrates how the wireless gum or tissue numbing device fits onto a finger of the professional, in this case the index finger (although other fingers may be utilized). In some embodiments, the wireless gum or tissue numbing device 200 may comprise an ABS Plastic base plate piece or assembly 230 (which could be made from other materials), an on/off switch cover 240, an on-off switch assembly 241, a printed circuit board including an on-off switch along with control electronics 235, a rechargeable power source 215, a soft rubber cover 205, and/or a vibration motor assembly 210. In some embodiments, the wireless gum or tissue numbing device 200 may vibrate at specific frequencies when activated (e.g., turned on or turned off). In some embodiments, the base plate piece or assembly 230 may comprise cavities that receive the different components. In some embodiments, moving from a first end of the base plate piece or assembly to the second end, the base plate piece or assembly 230 may comprise a first cavity for the on-off switch cover 240, a second cavity for the printed circuit board 235, a third cavity for the rechargeable battery 215 and/or a fourth cavity for the vibration motor assembly 210. In some embodiments, other components or circuit boards may be utilized such as wireless transceiver chipsets, communication circuit boards, control circuit boards or chipsets for controlling operations for vibration motor assemblies 210 or other components. In these embodiments, the base plate piece or assembly 230 may include additional cavities or openings and/or differently shaped openings or cavities in a bottom surface of the base plate piece of assembly 230.

In some embodiments, the soft rubber cover 205 may be placed, attached, connected and/or coupled to the bottom of the base plate piece or assembly 205 to cover the components of the wireless gum or tissue numbing device, such as the PCB with control electronics 235, on-off switch 231, the rechargeable battery 215, and/or the vibration motor 210. In some embodiments, the rechargeable battery may be a lithium battery or a lithium polymer battery. In some embodiments, the rechargeable battery 215 may be a 3.7 volt, 30 milliamp lithium battery. In some embodiments, the rechargeable battery may be square, rectangular and/or oval in shape. In some embodiments, the sides of the rechargeable battery may be less than 0.75 inches in length. In some embodiments, the sides of the rechargeable battery may alternatively be less than 0.64 inches in length. In some embodiments, other batteries may be utilized. In some embodiments, the rechargeable battery may have dimensions of 0.4 inch by 0.4 inch.

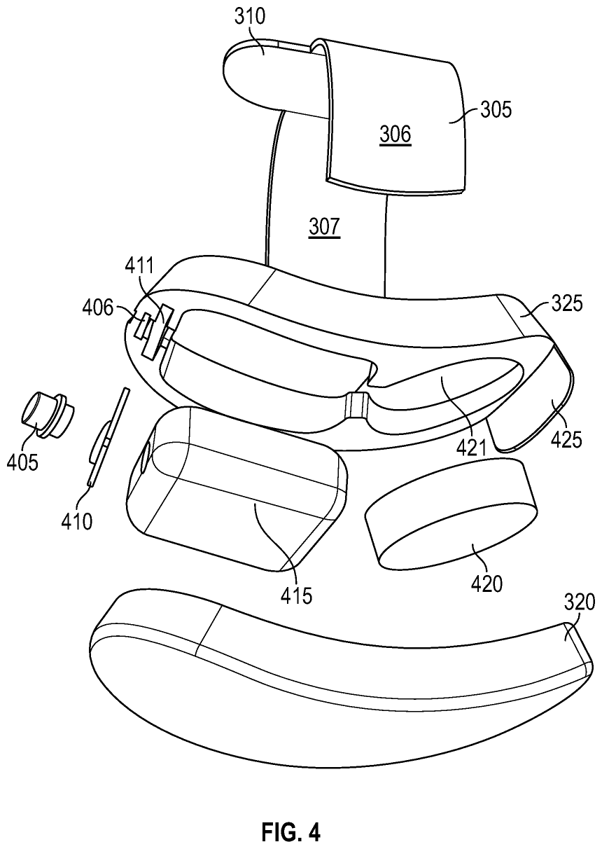

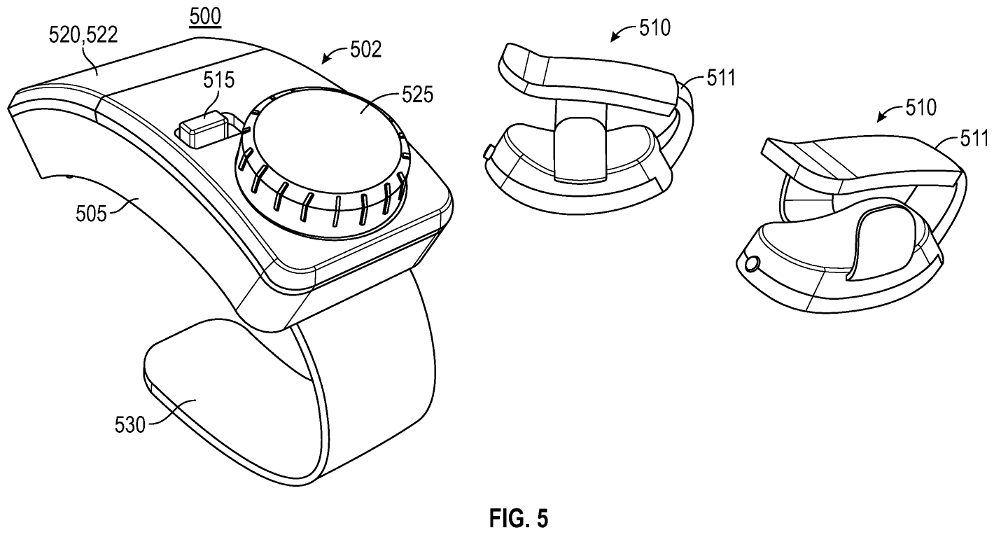

In some embodiments, the vibration motor may be a 3 Volt coin motor. In some embodiments, the vibration motor may be circular, square or oval in shape. In some embodiments, the vibration motor 210 may rotate at 12,000 revolutions per minute, or alternatively between 8,000 to 16,000 revolutions per minute. In some embodiments, the vibration motor 210 may be less than 0.75 inches in diameter, or alternatively less than 0.65 inches in diameter. In some embodiments, the vibration motor may be 0.4 inches in diameter. In some embodiments, the vibration motor 210 may have a low setting corresponding to 8,000 revolutions per minute; a medium setting corresponding to 10,000 revolutions per minute; and a high setting corresponding to 12,000 revolutions per minute. In some embodiments, a vibration motor assembly may have a maximum revolution per minutes (rpm) of 16,000 rpm, which may change the low, medium and high rpm settings. In some embodiments, the vibration motor assembly 210 may include a controller and/or control electronics and in other embodiments, the controller or control electronics may be located externally or apart from the coin motor. This is also true with respect to the wireless gum or tissue numbing device described below with respect to FIGS. 4 and 5. In some implementations, for example, the number of revolutions per minute for the vibration and/or motor assembly may range from 2,000 to 12,000 revolutions per minute. In some implementations, a voltage supplied to a motor controller and/or the vibration or motor assembly may be receive a voltage or voltage value and generate the number of revolutions per minute for the vibration/motor assembly based at least in part on the received voltage value or voltage. As an example, the received voltage value may be range from 1.0 to 5.0 volts and different voltage values may correspond to different revolutions per minute (e.g., 1.5 volts may correspond to 3,000 revolutions per minute, 2.5 volts may correspond to 6,000 revolutions per minute and 4 volts may correspond to 9,000 revolutions per minute. In some embodiments, no controller or control electronics may need to be utilized.

In addition to the wireless gum or tissue numbing device utilizing vibration to numb a patient's gum or tissue, the wireless gum or numbing device 200 may also utilize cold to numb a patient's gum or tissue. In some embodiments, the wireless gum or tissue numbing device 200 may utilize a cold surface in order to assist in numbing the tissue and/or gum. In some embodiments, the entire wireless gum or tissue numbing device 100 may be placed in a refrigerator in order to lower the temperature of the soft rubber cover 205 and assist in the numbing of the patient gum or tissue. In some alternative embodiments, the soft rubber cover 205 may be placed into a refrigerator or freezer in order to lower the temperature of the rubber cover 205 and also provide additional comfort to the patient. In these embodiments, the soft rubber cover 205 may be removed from the refrigerator and/or freezer, placed onto the wireless gum or tissue numbing device, and then positioned on the medical professional's finger.

In some embodiments, heat may be utilized to assist the medical professional in comforting the patient. In some embodiments, the whole wireless gum or tissue numbing device 200 cannot be placed into a microwave or oven without damaging the device and/or oven or microwave. In some embodiments, the soft rubber cover 205 may be placed into an oven or a slow heating device in order to heat up the cover. However, care must be taken to make sure the soft rubber cover 205 does not get too hot because that may damage a patient's gum or tissue. In some embodiments, a soft rubber cover 215 may also be made of ABS plastic with a secondary operation to cover it with soft rubber. After the software rubber cover 205 is heated, the cover 205 may be placed onto the wireless gum or tissue numbing device and provide additional comfort to the patient.

In some embodiments, the wireless gum or tissue numbing device 200 may be disposable after a single use or after a couple of uses. In some embodiments, if the wireless gum or tissue numbing device 200 is disposable, the software rubber cover 205 may not be removable. In such an embodiment, the wireless gum or tissue numbing device 200 may be sealed and/or may be water resistant.

In some embodiments, the rechargeable battery 215 may be utilized for two or three patients before needing to be recharged again. In some embodiments, the rechargeable battery 215 may be utilized for ten, twenty, fifty or a hundred patients or individuals. In some embodiments, the wireless gum or tissue numbing device may have a port that connects to the rechargeable battery 215 to allow an external power source to recharge the battery 215. In addition, there may be a pod device or plate device external to the gum or tissue numbing device where the rechargeable battery may be charged. In these embodiments, the battery 215 may be recharged via wireless charging or induction charging. In some embodiments, the rechargeable battery 215 may have a lifespan of 300 to 500 charge cycles and/or alternatively two to three years. In some embodiments, medical professionals may include have a number of rechargeable batteries on hand to be able to replace in case the rechargeable battery in the wireless gum or tissue numbing device loses its charge.

In some embodiments, in order to sterilize the wireless gum or tissue numbing device and/or the soft rubber cover 215, the wireless gum or tissue numbing device 200 and/or the soft rubber cover 215 may be exposed to ultraviolet light in order to kill bacteria and/or particles from the patient that may be present. This allows the wireless gum or tissue numbing device and/or the soft rubber cover 215 to be utilized multiple times. In other embodiments, a disinfecting spray may be utilized to sterilize the wireless gum or tissue numbing device and/or the soft rubber cover 215.

In some embodiments, a disposable soft rubber or a cloth cover may be utilized to protect the device from patient exposure or contamination. In such an embodiment, a wireless gum or tissue device may be reusable with the rubber covers being disposable. In some embodiments, this means that one or more soft rubber or cloth covers may also cover the entire assembly. In some embodiments, a latex, nitrate, or other protective barrier material utilized in the medical or dental industry may cover the wireless gum or tissue numbing device in order to provide sterilization for the patients.

In some embodiments, the wireless gum or tissue numbing device 200 may be operated as follows. In some embodiments, the wireless gum or tissue numbing device may have one vibration speed. In such an embodiment, a user may activate the device by pressing an on-off switch. In this embodiment, when the on-off switch is activated, the switch connects or couples the battery to the motor or vibration assembly and the wireless gum or tissue numbing device may vibrate until the on-off switch is touched again.

In some embodiments, the wireless gum or tissue numbing device may have multiple vibration speeds. In some embodiments, for example, the wireless gum or tissue numbing device may have a low vibration speed, a medium vibration speed or a high vibration speed. In this embodiment, a user may press the push button (or on-off switch) one time and the wireless gum or tissue numbing device may enter into a low vibration mode. In this embodiment, when the push button is pressed a second time, the wireless gum or tissue numbing device may enter into a medium vibration mode. In this embodiment, if the user presses the push button a third time, the wireless gum or tissue numbing device may enter into a high vibration mode. In this embodiment, if the user presses the push button a fourth time, the wireless gum or tissue numbing device may turn off or deactivate.

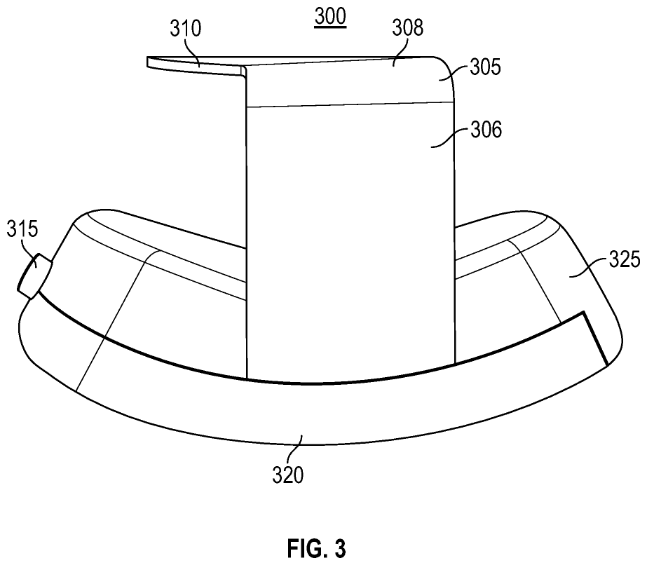

FIG. 3 illustrates an additional embodiment of the wireless gum or tissue numbing device according to some embodiments. In some embodiments, a wireless gum or tissue numbing device 300 may comprise a base assembly 325, an on/off switch 315, a soft rubber cover 320, a pulling or grip tab 310 and/or a finger retainer assembly 305. In some embodiments, the base assembly 325, the soft rubber cover 320 and/or the on/off switch 315 may operate as described above with the wireless gum or tissue numbing device described above with respect to FIGS. 1 and 2. In some embodiments, a finger retainer assembly 305 may have a semicircular shape or may be shaped like an arch. In some embodiments, a finger retainer assembly 305 may have two sides (side 306 is shown in FIG. 3 and both sides 306 and 307) and a top portion 308. In some embodiments, the first side 306 of the finger retainer assembly 305 and a second side 307 of the retainer assembly 305 may be attached, coupled, connected and/or attached into a corresponding first side and corresponding second side of the base assembly 325. In some embodiments, the first side 306 and the second side 307 of the retainer assembly may be connected, attached, coupled or inserted into corresponding sides of the base assembly 325 at a junction wherein the soft rubber cover 320 attaches to the base assembly 325. In some embodiments, the first side 306 and the second side 307 of the finger retainer assembly 305 rise above or and/or positioned vertically with respect to the base assembly 325 and are connected to the top portion 308 of the finger retainer assembly 308. In some embodiments, the top portion 308 of the finger retainer assembly may be slightly curved and then may be positioned or run in a horizontal direction across a top of a medical professional's finger. In some embodiments, a pulling or gripping tab 310 may allow easy positioning or removal of the wireless gum or tissue numbing device 300 on a medical professional's finger. In some embodiments, a pulsing or gripping tab 310 may have a semicircular shape.

In some embodiments, the pulling tab 310 may be connected and/or attached to a side of a top portion 308 of the finger retention assembly. In some embodiments, the first side 306, the second side 307 and the top portion 308 of the finger retainer assembly 305 are formed as one piece. In some embodiments, the first side 306, the second side 307, the top portion 308, and the pulling tab 310 of the finger retainer assembly 305 are formed as one piece. In some embodiments, the finger retainer assembly 305 is made of a soft rubber material so as to not irritate a patient's mouth or tissue as well as to not irritate or rub against a medical professional's finger. In some embodiments, the finger retainer assembly 305 may have a height of 0.5 inches, or alternatively may have a height in a range of 0.3 inches to 0.7 inches. In some embodiments, the finger retainer assembly 305 may have a width of 0.65 inches, or alternatively may have a range of width of 0.4 to 0.8 inches. In some embodiments, the finger retainer assembly 305 may be a depth from front to back of 0.35 inches, or alternatively may have a depth range of 0.2 to 0.5 inches. In some embodiments, the top portion 308 and the pulling tab 310 of the finger retaining assembly 305 may have a depth of front to back of 0.55 inches, or alternatively a depth range of 0.4 inches to 0.7 inches.

FIG. 4 illustrates an exploded view of an additional embodiment of the wireless gum or tissue numbing device according to some embodiments. In some embodiments, the wireless gum or tissue numbing device 400 may include the finger retaining assembly 305 and/or the base assembly 325. In some embodiments, the base assembly may include a bottom vertical plate 425 that is at a second end of the base assembly 325. In some embodiments, the bottom vertical plate 425 may be utilized to retain the soft cover assembly 320 at one end. In some embodiments, the bottom vertical plate 425 may be connected and/or attached to a bottom surface of the base assembly 325. In some embodiments, the bottom vertical plate 425 and the base assembly 325 may be formed as one piece. In some embodiments, the bottom portion of the base assembly 325 may include, from a first end to a second end, an on/off switch cavity 406, a control printed circuit board cavity 411, a rechargeable battery cavity 416, and/or a motor assembly cavity 421. In some embodiments, an on/off switch 405 may be placed and/or positioned in the on/off switch cavity 406. In some embodiments, a control PCB 410 may be placed and/or positioned in the PCB cavity 411. In some embodiments, a rechargeable battery 415 may be placed and/or positioned in a battery cavity 416. In some embodiments, the motor assembly 420 may be placed and/or positioned in the motor cavity 421. In some embodiments, the soft cover assembly 320 may hold and/or support these components (e.g., the PCB 410, the on/off switch 405, the battery 415 and/or the motor assembly 420) in their respective cavities. In some embodiments, the dimensions discussed above with respect to FIG. 2 may be the same as in the embodiment of FIG. 4. The additional components discussed above with respect to FIG. 2 also apply to the embodiment disclosed in FIG. 4.

In some embodiments, the wireless gum or tissue numbing device could include additional features and/or functions. However, in many embodiments, these features or functions may not be utilized because the wireless gum or tissue numbing device may be disposable and/or single use and the cost of the device may need to minimal. For example, the wireless gum or tissue numbing device may be voice activated by including software on the control PCB that responds to voice commands. In some embodiments, a pressure sensor and/or a piezo-electric sensor may be installed on the wireless gum or tissue numbing device may be activated in response to pressure on the sensor. In some embodiments, the wireless gum or tissue numbing device may further include a wireless communication transceiver, one or memory devices, one or more processors and/or software to receive commands from an external computing device in order to activate the wireless gum or tissue numbing device. The additional features or functions and/or electrical, mechanical or electro-mechanical components may be utilized in the embodiments described in FIGS. 2 and 4.

FIG. 5 illustrates a control assembly for one or more wireless gum and tissue numbing devices according to some embodiments. In some embodiments, a system may include a control assembly 500 and one or more wireless gum or tissue numbing device(s) 510 (there are two devices illustrated in FIG. 5). In some embodiments, the control assembly 500 may include one or more wireless communication transceivers 520 to control some or all of the operations of the one or more wireless gum and tissue numbing devices 510 that may include one or more wireless communication transceivers 511. In some embodiments, the one or more wireless communication transceivers 511 may communicate with and/or activate the one or more motor assemblies in the respective one or more gum and tissue numbing devices 510. In some embodiments, the control assembly 510 may include a connector assembly or wrist connection assembly or wristband 530 and a body 505, where the connection assembly 505 allows the body 505 to be placed on a user's wrist or arm. At one end, the connection assembly 530 may be connected, coupled or attached to the body 505. In some embodiments, the body may include a voltage regulator 525, an on/off switch 515, one or more power sources 522 and/or one or more wireless communication emitters and/or transceivers 520.

In some embodiments, the voltage and/or rpm regulator 525 may allow a user to set an rpm level for the one or more wireless gum or tissue numbing devices 510. In some embodiments, this means that a specific voltage level may be transmitted or communicated to the one or more gum or tissue numbing devices 510 via the one or more wireless communication transceivers 520. In some embodiments, the voltage and/or rpm regulator 525 may be a knob that may be moved to different settings. In some embodiments, the voltage and/or rpm regulator 525 may be a switch. In some embodiments, the voltage and/or rpm regulator 525 may include a microphone that receives voice commands and/or software that analyzes the voice command in order to determine which rpm setting the user or customer would like the one or more wireless gum or tissue numbing devices 510 to operate at. In some embodiments, the on/off switch 515 may allow a user or customer to turn on or off the control assembly 500 in order to activate the control assembly. In some embodiments, the on/off switch 515 may be a push button switch, although in alternative embodiments, the on/off switch may be a different type of switch or a knob. In alternative embodiments, the turning on and/or off of the control assembly may be voice controlled.

In some embodiments, the power source may be a battery 522. In some embodiments, the body 505 may have integrated therein a rechargeable battery and/or a replaceable battery 522. In some embodiments, the body 505 may have integrated therein a disposable battery 522. In some embodiments, the body may include a port or interface in order to recharge the battery 522. In some embodiments, the battery 522 may provide power for the on/off switch 515, the rpm or voltage regulator 525, and/or the one or more wireless communication transceivers or emitters 520. In some embodiments, the body 505 may include a one or more wireless communication transceivers or emitters integrated therein. In some embodiments, the one or more wireless communication transceivers or emitters 520 may operate according to the Bluetooth protocol, or in alternative embodiments, according to another personal area network protocol (e.g., Zigbee or others). In some embodiments, the one or more wireless communication transceivers 520 may operate according to wireless local area network communication protocols (e.g., such as WiFi or other 802.11 wireless protocols).

In some embodiments, the body 505 may include one or more controllers, one or more memory devices and/or computer-readable instructions stored in the one or more memory devices. In some embodiments, the voltage and/or rpm regulator 525 may generate a signal, command, instruction and/or message indicating a desired rpm for the motor assembly in the one or more wireless gum or tissue numbing devices. In some embodiments, the computer-readable instructions may be executable by the one or more controllers or processors and may communicate or send the generated desired rpm indication to the internal wireless communication transceiver or emitter 520, which in turn may communicate to the one or more wireless communication transceivers or receivers 511 in the one or more gum or tissue numbing devices 510. In some embodiments, this allows an operator to utilize the control assembly 500 to set operational characteristics for the one or more gum or tissue numbing devices 510 easily because the control assembly is on the operator or user's wrist. In some embodiments, the user or operator may then turn off the control assembly or device 500 and pickup the needle that may be utilized to apply the gum or tissue numbing substance to the gum or tissue. In some embodiments, the control assembly or device 500 may not need to be turned off when operating the one or more wireless gum or tissue numbing device. The user or operator may then place the one or more wireless gum or tissue numbing devices 510 in the desired area to initially numb the gum or tissue and the one or more wireless gum or tissue numbing devices may be operating at the desired rpm. In some embodiments, the signals, commands, instructions and/or messages may be communicated to the one or more wireless communication transceivers or receivers 511 in the one or more gum or tissue numbing device, which may communicate this information, parameters and/or data to the vibration or motor assembly in the one or more gum or tissue numbing device. In some cases, the vibration and/or motor assembly may include a controller to receive a message, information, parameter and/or a signal from the wireless communication transceiver or receiver 511. In some implementations, the one or more motor assemblies may be the motor assemblies or vibration assemblies described in FIGS. 2 and 4.

As detailed above, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “controller,” “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. For example, one or more of the devices recited herein may receive image data of a sample to be transformed, transform the image data, output a result of the transformation to determine a 3D process, use the result of the transformation to perform the 3D process, and store the result of the transformation to produce an output image of the sample. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.”

The processor or controller as disclosed herein can be configured with instructions to perform any one or more steps of any method as disclosed herein.

As used herein, the term “or” is used inclusively to refer items in the alternative and in combination.

As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A wireless gum numbing device, comprising
an on or off switch;
a soft rubber cover;
a base assembly, the soft rubber cover positioned on an underside of the base assembly;
two lateral support assemblies, the two lateral side assemblies to rise horizontally from a left side and a right side of the base assembly and to hold a user's finger from moving laterally during use of the wireless gum numbing device;
a finger holding clip, the user's finger touching a bottom side of the finger holding clip; and
a fingertip retention piece connecting the base assembly to the finger holding clip and to prevent the fingertip from moving too far in a forward direction.

2. The wireless gum numbing device of claim 1, wherein the base assembly has a plurality of recessed areas to hold a plurality of electronic components.

3. The wireless gum numbing device of claim 2, wherein a first recessed area of the plurality of recessed areas includes a vibration motor assembly to vibrate the soft rubber cover to generate a vibration sensation on a person's gum.

4. The wireless gum numbing device of claim 3, wherein a second recessed area of the plurality of recessed areas includes a rechargeable battery to provide power to the vibration motor assembly.

5. The wireless gum numbing device of claim 4, wherein a third recessed area off the plurality of recessed areas includes a control printed circuit board to control activation of the vibration motor assembly.

6. The wireless gum numbing device of claim 5, wherein a fourth recessed areas includes a switching assembly, a portion of the switching assembly exiting or being outside a rear portion of the base assembly.

7. A wireless gum numbing device, comprising
an on or off switch;
a soft rubber cover;
a base assembly, the soft rubber cover positioned on an underside of the base assembly;
a finger retaining assembly including a pulling tab assembly, the finger retaining assembly having an arch shape and rising vertically from a left side and a right side of the base assembly, the finger retaining assembly holding a user's finger in place during use of the wireless gum numbing device.

8. The wireless gum numbing device of claim 7, wherein the base assembly has a plurality of recessed areas to hold a plurality of electronic components.

9. The wireless gum numbing device of claim 8, wherein a first recessed area of the plurality of recessed areas includes a vibration motor assembly to vibrate the soft rubber cover to generate a vibration sensation on a person's gum.

10. The wireless gum numbing device of claim 9, wherein a second recessed area of the plurality of recessed areas includes a rechargeable battery to provide power to the vibration motor assembly.

11. The wireless gum numbing device of claim 10, wherein a third recessed area off the plurality of recessed areas includes a control printed circuit board to control activation of the vibration motor assembly.

12. The wireless gum numbing device of claim 11, wherein a fourth recessed areas includes a switching assembly, a portion of the switching assembly exiting or being outside a rear portion of the base assembly.

13. A wireless gum numbing system, comprising:
a wrist-based control assembly or device; and
two receiving wireless gum numbing devices, each of the two receiving wireless gum numbing devices having a vibration motor assembly and a wireless communication transceiver, wherein:
the wrist-based control assembly includes a transmitter or transceiver, the transmitter or transceiver to wirelessly communicate a signal to the two receiving wireless gum numbing devices, the signal including data related to activating the vibration motor assembly and a speed of vibration/rotation for the vibration motor assembly.

14. The wireless gum numbing system of claim 13, the wrist-based control assembly including a body and a wristband connection assembly coupled to an underside of the body, the body including a voltage revolution per minute (rpm) regulator to control the speed of vibration of the vibration motor assembly, an on/off switch to control operation of the vibration motor assembly in each of the receiving wireless gum numbing devices, and a power source to provide power to the transmitter and other electronic components integrated within the body.

15. The wireless gum numbing system of claim 14, each of the wireless gum numbing devices including:
an on or off switch;
a soft rubber cover;
a base assembly, the soft rubber cover positioned on an underside of the base assembly; and
a finger retaining assembly including a pulling tab assembly, the finger retaining assembly having an arch shape and rising vertically from a left side and a right side of the base assembly, the finger retaining assembly holding a user's finger in place during use of the wireless gum numbing device.
wherein the base assembly has a plurality of recessed areas to hold a plurality of electronic components.

16. The wireless gum numbing device of claim 15, wherein a first recessed area of the plurality of recessed areas includes a vibration motor assembly to vibrate the soft rubber cover to generate a vibration sensation on a person's gum.

17. The wireless gum numbing device of claim 16, wherein a second recessed area of the plurality of recessed areas includes a rechargeable battery to provide power to the vibration motor assembly.

18. The wireless gum numbing device of claim 19, wherein a third recessed area off the plurality of recessed areas includes a control printed circuit board to control activation of the vibration motor assembly.

19. The wireless gum numbing device of claim 18, wherein a fourth recessed areas includes a switching assembly, a portion of the switching assembly exiting or being outside a rear portion of the base assembly.
